(19) United States
(12) Patent Application Publication
(10) Pub. No.: US 2024/0058435 A1
(43) Pub. Date: Feb. 22, 2024

(54) SUSTAINED RELEASE INACTIVATED VACCINES

(71) Applicant: Trevor Percival Castor, Arlington, MA (US)

(72) Inventor: Trevor Percival Castor, Arlington, MA (US)

(73) Assignee: Aphios Corporation, Arlington, MA (US)

(21) Appl. No.: 18/269,553

(22) PCT Filed: Nov. 22, 2021
(86) PCT No.: PCT/US2021/060407

§ 371 Date: Jun. 24, 2023

Related U.S. Application Data
(60) Provisional application No. 63/116,902, filed on Nov. 22, 2020.

(51) Int. Cl. A61K 39/215; A61K 39/145; A61K 39/21; A61K 9/51; A61K 39/07; A61P 31/18; A61P 31/04

(52) U.S. Cl. CPC A61K 39/215; A61K 39/145; A61K 39/21; A61K 9/5153; A61K 39/07; A61P 31/18; A61P 31/04; A61K 2039/5252

(57) ABSTRACT

The present invention is directed to methods and apparatus for creating a sustained release pathogen vaccine for COVID-19, influenza, HIV and other infectious human and animal viruses and pathogens using supercritical, critical, or near-critical fluids with or without polar cosolvents for simultaneously inactivating virions and pathogens, and encapsulating the inactivated virions and pathogens in biodegradable polymer nanospheres for administration to a patient. The present invention continuously inactivates SARS-CoV-2, influenza, HIV and other infectious human and animal viruses and pathogens, and nanoencapsulates the inactivated virions and pathogens in biodegradable polymer nanospheres to provide a safe and effective sustained-release vaccine, especially for the frail and elderly.

### FIELD OF INVENTION

The present invention is directed to methods and apparatus for creating a sustained release vaccine for virus and other pathogenic diseases using supercritical, critical, or near-critical fluids for simultaneously inactivating virions and other pathogen particles and encapsulating the inactivated particles in biodegradable polymer nanospheres. The present invention is directed to methods and apparatus for creating sustained release vaccines for COVID-19, influenza, HIV and other infectious human and animal viruses and pathogens.

### REFERENCES TO OTHER PATENTS

This application discloses a number of improvements and enhancements to the method for making inactivated viruses for use in vaccines as disclosed in U.S. Pat. No. 7,033,813 to Castor et al., which is hereby incorporated by reference in its entirety.

### BACKGROUND OF THE INVENTION

There is a vital and pressing need to develop effective vaccines for COVID-19, influenza, HIV and other infectious human and animal viruses and pathogens. Most of the vaccines under study include traditional recombinant proteins, replicating and non-replicating viral vectors, and nucleic acid and mRNA approaches. Each of these vaccine platforms has advantages and limitations. About 50% of commercial viral vaccines, including influenza, are inactivated. No single platform is likely to meet global demand [Corey et al., 2020] for effective viral vaccines.

Seasonal Influenza is a common and serious respiratory illness that contributes to the reduction of quality of life and a significant loss of manpower hours each year. Seasonal influenza epidemics cause 3 million to 5 million severe cases, and 300,000 to 500,000 deaths globally each year, according to the World Health Organization (WHO). In the United States, there are 140,000 to 710,000 influenza-related hospitalizations and 12,000 to 56,000 deaths each year, with the highest burden of disease affecting the very young, the very old and people with co-existing medical conditions according to the Center for Disease Control.

In 2017, there was widespread flu activity in 46 states. About 80% of flu cases were H3N2, a strain of seasonal influenza A virus that tends to be more deadly in young children and the elderly than its influenza A H1N1 counterpart and influenza B strains that appear with more frequency later in the flu season (Zimmerman, 2018). A few years ago, and still today, there are also grave concerns about the avian flu (H5N1) circulating in the Far East and traveling with migratory birds to the west. There are significant concerns that the H5N1 avian flu strain, if mutated for human-to-human transfer, and/or the H1N1 swine flu strain, if mutated into a more virulent form, could result in an influenza pandemic of the magnitude of the 1918 outbreak that killed over 40 million people worldwide; the mortality rate is about 50% for individuals infected with H5N1 through bird to man transmissions and, while the mortality rate of the H1N1 swine flu strain is lower, its human-human transmission efficiency is much higher.

Vaccination against the flu is only partially effective even when influenza vaccines are well-matched to circulating viruses, 40% to 60% depending on the population demographics, which is lower than that of most licensed non-influenza vaccines. For the 2016-2017 season, even though the Northern hemisphere vaccine was updated to include the H3N2 virus strain, the preliminary estimate of vaccine effectiveness is 42% overall and only 34% against influenza H3N2 virus—some of these effects can be mitigated by using adjuvants or high dose vaccines to generate more robust immune responses in the elderly (Paules et al., 2018). In addition, public participation in the vaccination program is far from complete, even among high-risk individuals such as the elderly, healthcare workers, and children. Furthermore, production of the vaccine is not without problems. Difficulties with the manufacturing (some strains are difficult to grow in chicken eggs, which is the currently approved preparation method) and distribution of the vaccine could lead to delays in receiving the vaccine until well into the flu season. Finally, the three strains of influenza that comprise the vaccine are predicted each year based on surveillance reports from the previous flu season. Should a different strain emerge, such as the A/Fujian strain that arose earlier in 2003, vaccinated individuals may not be protected. In fact, the effectiveness of the 2003 influenza vaccine was predicted to be as low as 14-60% instead of the more typical 60-80% because of the A/Fujian strain (Ault, 2004). Anti-influenza drugs, effective in reducing the severity and length of the disease, are therefore an important adjunct to the vaccine. Only four anti-influenza drugs are available in the United States today; two of these drugs are effective against influenza A (Centers for Disease Control and Prevention, 2001c). Due to the ever-changing properties of the influenza virus, development of resistant strains is a problem, requiring the continued development of new drugs. In addition, the threat of an epidemic is ever-present; the emergence and spread of avian influenza strains in Mexico (H1N1), Hong Kong (H5N1) and the Netherlands (H7N7) are particularly worrisome in causing a new pandemic. Such a pandemic could rapidly spread in today's world of high-speed travel and the close confines of urban living.

The aged population of the United States (age 65+) now exceeds 47.8 million (2015 census) and this number is projected to more than double to 98.2 million by 2060. Aged individuals are at increased risk of severe and often mortal complications from influenza. Annual vaccination with the trivalent influenza vaccine is highly recommended for this at-risk group. In the elderly, vaccination is only 40-60% effective in preventing influenza-related hospitalizations and death; the vaccine is only 30-50% effective in preventing influenza-like illness in frail elderly. By comparison, the flu vaccine is about 70-90% effective in preventing illness in healthy young adults. The reduced efficacy of influenza vaccines in the elderly is likely due to reduced immune responses to vaccination in this age group.

Studies in elderly have shown reduced cellular and humoral immunity following exposure to the traditional intramuscular delivery of aqueous influenza antigens compared to young adults. Therefore, the development of novel delivery systems to improve persistence of antigen availability of influenza vaccines would improve immune responsiveness and efficacy, particularly for the elderly.

The reduced efficacy of influenza vaccines in the elderly is likely due to reduced immune responses to vaccination in this age group. Studies in elderly have shown reduced cellular and humoral immunity following exposure to traditional intramuscular delivery of aqueous influenza antigens compared to younger adults. Therefore the development of novel delivery systems to improve the immune response to influenza vaccines in the elderly are highly desirable.

The novel coronavirus, COVID-19, emerged in Wuhan, China in late December 2019. Coronaviruses are a large family of viruses that may cause illness in animals and humans. In humans, several coronaviruses are known to cause respiratory disease such as Middle East Respiratory Syndrome (MERS), Severe Acute Respiratory Syndrome (SARS) and the most recently discovered COVID-19. These viruses are all genetically related with both SARS (10% fatality rate) and MERS (37.4% fatality rate) being more deadly than COVID-19 but much less infectious. Should the next coronavirus be as infectious as COVID-19 but have a fatality rate approaching SARS or worse yet MERS, this future pandemic would be much more devastating than the current COVID-19 pandemic unless we have the tools and capabilities in place to contain and manage coronaviruses. Industry and governments have responded to the current pandemic with an urgent search for vaccines and testing of extant malaria, HIV and Ebola antiretrovirals against COVID-19.

Most of the vaccines under study include traditional recombinant proteins, replicating and non-replicating viral vectors, and nucleic acid and mRNA approaches. DNA vaccines require electroporation to facilitate DNA entry into cells and mRNA vaccines typically use lipid nanoparticles to protect and deliver the mRNA. Recombinant vaccines that express the spike protein utilize viral vectors such as adenovirus for which a significant amount of the population harbors neutralizing antibodies. The University of Oxford, partnered with Astra Zeneca, has entered clinical trials with a recombinant chimpanzee adenovirus previously utilized for developing an Ebola vaccine.

Each of these vaccine platforms has advantages and limitations. For example, the scalability and temperature stability of mRNA vaccines are of concern. Moderna's mRNA vaccine has been heralded as the vaccine platform of great promise with significant funding by BARDA. There are, however, no mRNA vaccines approved by the FDA for an infectious disease. Thus, as earlier stated, there is an urgent need to increase the availability of vaccine platforms since “no single platform is likely to meet global demand” [Corey et al., 2020]. The overall goal of this invention is a safe and effective extended-release vaccine against COVID-19. The current COVID-19 pandemic is having a significant impact on the morbidity and mortality of infected patients and is a threat to the health and welfare of United States citizens and citizens of other countries around the world. The COVID-19 pandemic is also having a significant impact on the economies and social fabric of all societies around the world.

Coronaviruses constitute the subfamily Orthocoronavirinae, in the family Coronaviridae, order Nidovirales, and realm Riboviria [de Goot et al., 2011]. They are lipid-enveloped viruses with a positive-sense single-stranded RNA genome and a nucleocapsid of helical symmetry. The genome size of coronaviruses ranges from approximately 27 to 34 kilobases, the largest among known RNA viruses [Sexton et al., 2016]. The diameter of the virus particles is about 120 nm [Fehr et al., 2015]. The envelope of the virus in electron micrographs appears as a distinct pair of electron dense shells (FIG. 1) [Neuman et al., 2006] with well-defined spikes. Infection begins when the virus enters the host organism and the spike protein attaches to its complementary host cell receptor. After attachment, a protease of the host cell cleaves and activates the receptor-attached spike protein. Depending on the host cell protease available, cleavage and activation allows cell entry through endocytosis or direct fusion of the viral envelop with the host membrane [Simmons et al., 2006].

There is an urgent need for improved COVID-19 vaccines for immunocompetent individuals, and patients with compromised immune systems. The immunocompetent individuals most of whom show minor or no symptoms still need to be immunized to prevent them from becoming symptomless carriers and spread the disease to susceptible individuals. It is estimated that anywhere from 25-50% of the affected individuals are shedding virus while being free of symptoms. While there are other issues of significance such as recombinant versus natural antigens versus nucleic acid delivery systems, there will remain a need for the improved delivery of COVID-19 vaccine antigens to produce an improved immunogenic response especially with the elderly. The aged population of the United States (age 65+) now exceeds 47.8 million (2015 census) and this number is projected to more than double to 98.2 million by 2060. Aged individuals are at increased risk of severe and often mortal complications from COVID-19.

A sustained release COVID-19 vaccine would be very impactful to the millions of severely infected individuals and prevent thousands of deaths. Backup vaccine platforms are badly needed.

Since the start of the HIV/AIDS pandemic in 1982, over 32 million people have died from AIDS and there were approximately 37.9 million people living with HIV in 2018. In the United States, an estimated 1.1 million people are currently living with HIV and approximately 40,000 new infections occur each year. These individuals, even when vaccinated, are more susceptible to the SARS-CoV-2 virus and its variants. In 2017, there were ˜17,000 deaths in the US from a Stage 3 AIDS diagnosis and ˜16,000 deaths among people diagnosed with HIV in 2016. Although ART is undoubtedly a life-saving therapy for millions of AIDS patients, the persistence of latent HIV-infected cellular reservoirs represents the major hurdle to virus eradication, since latently infected cells remain a permanent source of viral reactivation. Recently, there have been significant advances in the cure of HIV-1 in chronically infected mice using CRISPR Cas9 controls with and without ART. These advances however, face significant hurdles in advancing to the clinic and HIV-1 patients. They may also require purging of latent reservoirs such as the brain that is protected by the blood brain barrier. There are no commercially available drugs for treating HIV latency. Additional strategies are needed to purge and eliminate latently infected cells from HIV infected individuals. In addition to antiviral and latency reversal therapeutics, a sustained release inactivated HIV vaccine could be used therapeutically as the immunological component of a ‘kick and kill’ strategy. A sustained release inactivated HIV vaccine can also be used independently as a therapeutic and as a prophylactic.

The gravity of the epidemiological situation will make any efficient vaccine a highly attractive product, even if it may require an annual boost for maintenance of protection; based on contemporary scientific data, such a scenario is likely. The uses of a whole inactivated vaccine include (1) being part of a vaccine regimen using both DNA or viral vector and a whole inactivated vaccine as the boost for sterilizing immunity; (2) use of the whole inactivated vaccine for therapeutic purposes—especially in cases where HAART has failed and (3) replacement of HAART for the whole inactivated vaccine for use in third world countries that cannot afford the very expensive drugs. The necessary vaccine strategy for HIV may resemble the situation that currently exists with influenza vaccination where annual shots of an inactivated vaccine that targets the predominant viral strain, which differs from year to year, are needed.

A whole-killed inactivated HIV vaccine preparation may become a valuable component for such vaccination regimen. Historically, killed HIV vaccines did not exhibit strong immunogenicity and protective efficacy for HIV infection since the thermal or chemical means of inactivation resulted in total or near-total disruption of virion structure, in particular of the denaturation of surface proteins. These concerns are addressed by a novel virus inactivation technology that employs materials known as SuperFluids or SFS which are supercritical, critical or near-critical fluids with or without polar cosolvents or entrainers and their mixtures.

SuperFluids are used to continuously inactivate SARS-CoV-2 influenza, HIV, and other infectious human and animal viruses and pathogens and nanoencapsulate the inactivated virions and pathogens in biodegradable polymer nanospheres to sustain their release. Two SuperFluids™ technology platforms, CFI™ and PNS™ are utilized.

The CFI process is purely physical and does not require post-processing to remove chemicals. We have demonstrated that the CFI™ process inactivates both enveloped viruses such as Influenza, MuLV, VSV, Sindbis, HIV, TGE, and BDVD, and the non-enveloped viruses such as Polio, Adeno, EMC, Reo, and Parvo, while preserving biological activity [Castor et al., 1999-2002; Castor, 2001]. We have developed a single-stage laminar flow process and defined operating conditions for inactivating >6 logs of Influenza, HIV and other enveloped viruses in times less than 20 secs with high retention of antigenic and immunogenic properties [Castor et al., 2006]. We have also demonstrated that levels of inactivation are incrementally increased by the addition of stages. Theoretically, >30 logs of inactivation of enveloped viruses such as coronavirus can be achieved in a 5-stage laminar flow CFI unit.

In PNS™ (polymer nanospheres) technology, a biodegradable polymer is dissolved in SuperFluids™, mixed with the target therapeutic and decompressed through a nozzle into a biocompatible buffer. As a result of decompression, the hydrophobic polymer comes out of solution and thermodynamically self-assemblies into nanoparticles encapsulating the therapeutic target. In collaboration with the Defense Science and Technology Laboratory (DSTL), an executive agency of the Ministry of Defense of the United Kingdom, we demonstrated that PNS nanoparticles of recombinant protein antigen (rPa) were immunogenic and fully protective in an anthrax-mice challenge study [unpublished data]. PNS has also been used to nanoencapsulate biosynthetic insulin making it orally bioavailable in a rat model of diabetes [Castor, 2013] and Bryostatin-1 making it orally bioavailable as an Alzheimer' disease therapeutic [Castor et al., 2015].

We achieve the simultaneous inactivation and nanoencapsulation by utilizing SuperFluids™ enriched with biodegradable polymers to penetrate SARS-CoV-2 influenza, HIV and other infectious human and animal viruses and pathogens under moderate conditions of temperature and pressure to inactivate the virions and pathogens as a result phase change from a fluid to a gas during decompression, and to nanoencapsulate the inactivated virions and pathogens in polymer nanospheres as a result of thermodynamically-driven self-assembly in an aqueous biocompatible buffer to create a safe, highly-immunogenic, sustained-release nanovaccines for COVID-19, influenza, HIV and other infectious human and animal viruses and pathogens.

### SUMMARY OF THE INVENTION

The present invention relates to the rapid, continuous-flow manufacturing of a sustained release inactivated vaccines for COVID-19, influenza, HIV and other infectious human and animal viruses and pathogens utilizing SuperFluids or SFS which are supercritical, critical or near-critical fluids with or without polar cosolvents or entrainers and their mixtures. SuperFluids of interest are fluids such as carbon dioxide, nitrous oxide and propane that are normally gaseous at ambient conditions of pressure and temperature. When compressed above their critical pressure and critical temperature, they become dense phase fluids with enhanced thermodynamic properties of solvation, penetration, selection and expansion.

Embodiments of the present invention are directed to methods and apparatus for using SuperFluids to make inactivated and nanoencapsulated vaccines against COVID-19, influenza, HIV and other infectious human and animal viruses and pathogens.

The present invention relates to methods and apparatus for the inactivation of virions and pathogens by SuperFluids containing hydrophobic polymers in a process called CFI™ (critical fluid inactivation). In CFI, SuperFluids are used to penetrate and inflate virion particles. Upon decompression, the rapidly expanding SFS disrupts the overinflated virion particles which are inactivated as a result of single-point rupture. The CFI process is purely physical and does not require post-processing to remove chemicals. The inventor has demonstrated that CFI inactivates both enveloped viruses such as Coronaviruses, Influenza, MuLV, VSV, Sindbis, HIV, TGE, and BDVD, non-enveloped viruses such as Polio, Adeno, EMC, Reo, and Parvo and bacterial pathogens such as E. coli and Bacillus subtilis, while preserving antigenicity and immunogenicity.

The present invention relates to methods and apparatus wherein the inactivated virions are encapsulated in polymer nanospheres using a technology called PNS™. In the PNS technology, a biodegradable polymer is dissolved in SuperFluids, mixed with the target therapeutic and decompressed through a nozzle into a biocompatible buffer. The hydrophobic polymer comes out of solution and thermodynamically self-assembles into nanoparticles encapsulating the therapeutic target. The inventor has demonstrated that PNS nanoparticles of recombinant protein antigen (rPa) were immunogenic and fully protective in an anthrax-mice challenge study. PNS has also been used to nanoencapsulate biosynthetic insulin making it orally bioavailable in a rat model of diabetes and Bryostatin-1 making it orally bioavailable as an Alzheimer' disease therapeutic.

In one embodiment of the present invention, SuperFluids enriched with biodegradable polymers are utilized to penetrate the virions and pathogens under moderate conditions of temperature and pressure. This inactivates the virions as a result of phase changes from fluid to gas during decompression. The inactivated virions are nanoencapsulated into polymer nanospheres.

In one aspect, operating parameters and process conditions are provided for the continuous and simultaneous inactivation and nanoencapsulation of the virions and pathogens. The present invention involves methods of inactivation and nanoencapsulation, and the continuous integration of these two methods.

Preferably, the critical, near-critical or supercritical fluid is at a temperature in the range of 0° C. to 100° C. This temperature range is in a range in which proteins held in aqueous solutions do not denature. Preferably, the critical, near-critical or supercritical fluid has a temperature that does not exceed 60° C. And even more preferred, the critical, near-critical or supercritical fluid has a range of 4° C. to 40° C.

Preferably, the critical, near-critical or supercritical fluid has a pressure in which the admixture is made and maintained, which pressure is 0.75 to 20.0 times the critical pressure of the gas comprising such fluid.

A preferred fluid is selected from one or more of gases of the group consisting of fluorocarbons, such as chlorofluoromethanes, alkanes, such as ethylene, propane and ethane and binary gases such as nitrous oxide and carbon dioxide and their mixtures. Preferably, the critical, near-critical or supercritical fluid further comprises one or more modifiers selected from the group consisting of ethanol, methanol, acetone and ethylene glycol.

A particularly preferred critical, near-critical or supercritical fluid is nitrous oxide with trace quantities of carbon dioxide, in the range of 10 to 1,000 parts per million carbon dioxide at approximately 12° C. to 40° C. and 800 to 5,000 psig; and, even more preferred, nitrous oxide with trace quantities of carbon dioxide, in the range of 10 to 1,000 parts per million carbon dioxide at approximately 16° C. to 26° C. and 1,600 to 5,000 psig; and, most preferred, nitrous oxide with trace quantities of carbon dioxide, in the range of 10 to 1,000 parts per million carbon dioxide at approximately 22° C. and approximately 3,000 psig. At these conditions, more than 3 logs of HIV virus can be inactivated in a single-stage or single-pass processing unit.

A particularly preferred critical, near-critical or supercritical fluid is nitrous oxide at approximately 12° C. to 40° C. and 800 to 5,000 psig; and even more preferred, nitrous oxide at approximately 16° C. to 26° C. and 1,600 to 5,000 psig; and most preferred, nitrous oxide at approximately 22° C. and approximately 3,000 psig. At these conditions, proteins show little change in function.

A particularly preferred critical, near-critical or supercritical fluid is chlorofluoromethane at approximately 10° C. to 40° C. and 1,000 to 5,000 psig; and, even more preferred, chlorofluoromethane at approximately 22° C. and 2,000 to 4,000 psig.

One embodiment of the present invention features an apparatus for inactivating one or more virions in a sample of virions and pathogens and nanoencapsulated the inactivated virions and pathogens in biodegradable polymer nanospheres. The apparatus comprises a vessel for forming an admixture of virions and pathogens sample with hydrophobic polymers dissolved in a critical, near-critical or supercritical fluid, which critical, near-critical or supercritical fluid that is capable of being received by one or more virions associated with the sample. Upon removal of the critical, near-critical or supercritical fluid one or more virions are inactivated and nanoencapsulated. The apparatus further comprises depressurization means for removing the critical, near-critical or supercritical fluid to render one or more virions inactive while retaining the constituents of the virus in the sample antigenic and immunogenic and encapsulated in biodegradable polymer nanospheres.

Preferably, the vessel is in communication with a continuous supply of the virions and pathogens sample. In addition, the depressurization means is capable of receiving a continuous supply of the admixture of the virions and pathogens sample and the critical, near-critical or supercritical fluid.

Preferably, the vessel retains the admixture for a period of time to result in a thousand-fold to four-thousand-fold reduction of active virions and pathogens. In addition, more preferably, the vessel retains the admixture for a period of one to thirty minutes.

Vaccines made from inactivated viruses have proven to be safe and effective. The present invention continuously inactivates the SARS-CoV-2 virus and nanoencapsulate the inactivated coronavirus virions in biodegradable polymer nanospheres, providing an effective sustained-release vaccine.

The present invention is significant because traditional vaccines are immediate release vaccines. Extended release of antigens can significantly improve immune response of the very young and elderly. Improved, sustained release influenza and COVID-19 vaccines are very impactful to the elderly and millions of severely infected individuals, preventing thousands of deaths in the United States and globally.

These and other features, aspects and advantages of the present teachings are better understood with reference to the following description, examples, and appended claims.

### BRIEF DESCRIPTION OF THE DRAWINGS

FIG. 1 shows a scanning electron microscopic image of coronavirus;

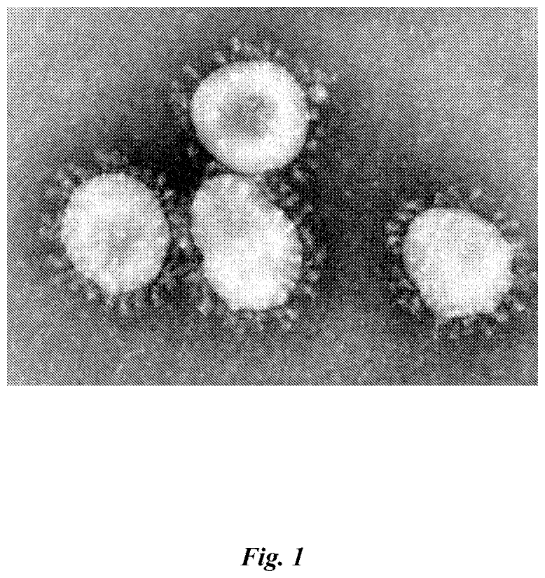

FIG. 2 shows before-and-after photomicrographs of normal viral activity before CFI™ and disrupted and inactivated viral activity after CFI™ treatment;

FIG. 3 schematically illustrates the SuperFluids™—Polymer Nanosphere (SFS-PNS) process according to the present invention;

FIG. 4 is a photomicrograph showing rPA-02 nanospheres;

FIG. 5 shows (a) single-stage and (b) multiple-stage SuperFluids™ viral inactivation devices;

FIG. 6 is a process flow diagram for a two-stage CFI™/PNS™ unit according to the present invention;

FIG. 7 schematically illustrates a PNS pilot plant according to the present invention; and

FIG. 8 shows CFI-treated HIV generates a humoral immune response in mice.

### DETAILED DESCRIPTION OF THE PREFERRED EMBODIMENTS

The present invention is described in detail as methods and apparatus for inactivating one or more virions and pathogens, and nanoencapsulating the inactivated virions and pathogens in biodegradable polymer nanospheres to sustain their release. The methods and apparatus utilize SuperFluids or SFS which are supercritical, critical or near-critical fluids with or without polar cosolvents or entrainers and their mixtures. SuperFluids of interest are fluids such as carbon dioxide, nitrous oxide and propane that are normally gaseous at ambient conditions of pressure and temperature. When compressed above their critical pressure and critical temperature, they become dense phase fluids with enhanced thermodynamic properties of solvation, penetration, selection and expansion.

A material becomes a critical fluid at conditions that equal its critical temperature and critical pressure. A material becomes a supercritical fluid at conditions that exceed both its critical temperature and critical pressure. The parameters of critical temperature and critical pressure are intrinsic thermodynamic properties of all sufficiently stable pure compounds and mixtures. Carbon dioxide, for example, becomes a supercritical fluid at conditions that exceed its critical temperature of 31.1° C. and its critical pressure of 72.8 atm (1,070 psig). In the supercritical fluid region, normally gaseous substances such as carbon dioxide become dense phase fluids that have been observed to exhibit greatly enhanced solvating power. At a pressure of 204 atm (3,000 psig) and a temperature of 40° C., carbon dioxide has a density of approximately 0.8 g/cc, compared with a density of 0.002 g/cc at standard conditions (0° C. and 1.0 atm), and behaves much like a nonpolar organic solvent, having a dipole moment of zero Debyes.

A supercritical fluid displays a wide spectrum of solvation power, as its density is strongly dependent upon temperature and pressure. Temperature changes of tens of degrees or pressure changes by tens of atmospheres can change a compound's solubility in a supercritical fluid by an order of magnitude or more. This feature allows for the fine-tuning of solvation power and the resulting fractionation of mixed solutes. The selectivity of nonpolar supercritical fluid solvents can also be enhanced by addition of compounds known as modifiers (also referred to as entrainers or cosolvents). These modifiers are typically polar organic solvents such as acetone, ethanol, methanol, methylene chloride or ethyl acetate. Varying the proportion of modifier allows wide latitude in the variation of solvent power.

In addition to their unique solubilization characteristics, supercritical fluids possess other physicochemical properties that add to their attractiveness as solvents. They can exhibit liquid-like density yet still retain gas-like properties of high diffusivity and low viscosity. The latter increases mass transfer rates, significantly reducing processing times. Additionally, the ultra-low surface tension of supercritical fluids allows facile penetration into microporous materials, increasing extraction efficiency and overall yields.

A material at conditions that border its supercritical state will have properties that are similar to those of the substance in the supercritical state. These near-critical fluids are also useful for the practice of this invention. For the purposes of this invention, a near-critical fluid is defined as a fluid which is (a) at a temperature between its critical temperature (Tc) and 75% of its critical temperature and at a pressure at least 75% of its critical pressure or (b) at a pressure between its critical pressure (Pc) and 75% of its critical pressure and at a temperature at least 75% of its critical temperature. In this definition, pressure and temperature are defined on absolute scales, e.g., Kelvin and psia, respectively. Table 1 shows how these requirements relate to some of the fluids relevant to this invention. To simplify the terminology, materials that are utilized under supercritical, near-critical conditions or exactly at their critical point with or without polar entrainers and their mixtures will be jointly referred to as “SuperFluids” or “SFS.”

TABLE 1
Physical Properties of SuperFluids (SFS)

| | | BP | Pᵥₐₚ | T_{c} | P_{c} | 75% of T_{c} | 75% of P_{c} |
|---|---|---|---|---|---|---|---|
| Fluid | Formula | (° C.) | (psia @ 25° C.) | (° C.) | (psia) | (° C.) | (psia) |
| Carbon dioxide | CO₂ | −78.5 | 860 | 31.1 | 1070 | −45.0 | 803 |
| Nitrous oxide | N₂O | −88.5 | 700 | 36.5 | 1051 | −41.0 | 788 |
| Propane | C₃H₈ | −42.1 | 130 | 96.7 | 616 | 4.2 | 462 |
| Ethane | C₂H₆ | −88.7 | 570 | 32.3 | 709 | −44.1 | 531 |
| Ethylene | C₂H₄ | −103.8 | NA | 9.3 | 731 | −61.4 | 548 |
| Freon 11 | CCl₃F | 23.8 | 15 | 198.1 | 639 | 80.3 | 480 |
| Freon 21 | CHCl₂F | 8.9 | 24 | 178.5 | 750 | 65.6 | 562 |
| Freon 22 | CHClF₂ | −40.8 | 140 | 96.1 | 722 | 3.8 | 541 |
| Freon 23 | CHF₃ | −82.2 | 630 | 26.1 | 700 | −48.7 | 525 |

BP = Normal boiling point;
Pᵥₐₚ = Vapor pressure;
T_{c} = critical temperature;
P_{c} = critical pressure

SuperFluids, when compressed, exhibit enhanced solvation, penetration and expansive properties. SuperFluids are utilized to penetrate and inflate viral particles. The overfilled particles are then decompressed and, as a result of rapid phase conversion, rupture at their weakest points. The aim is to introduce minimal controlled damage to the structure of the virion, rendering it non-infective. This will preserve its overall tertiary structure and, expose internal epitopes that are usually inaccessible to the immune system. This technique is purely physical, and does not rely on denaturing heat, chemicals or irradiation.

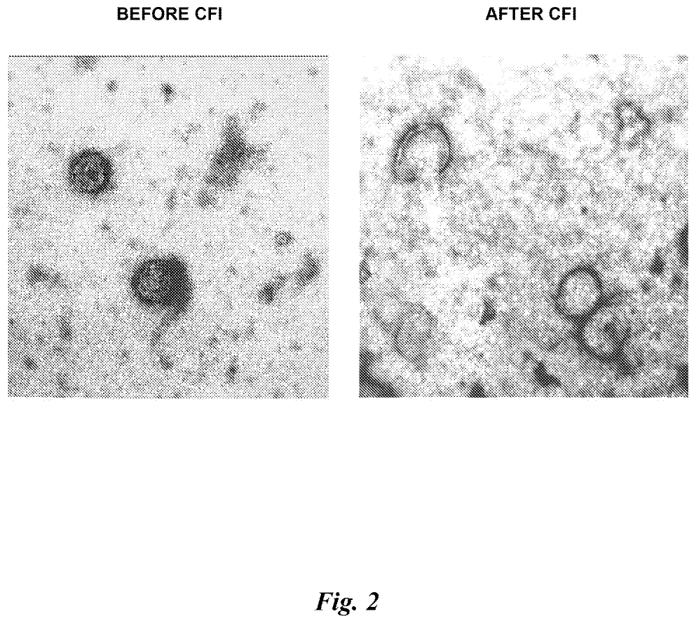

One embodiment of the invention utilizes SuperFluids as the preferred method of inactivation in a process called CFI™. SuperFluids™ (SFS) of interest are normally gases, such as carbon dioxide and nitrous oxide, at room temperature and pressure. When compressed, these gases become dense-phase fluids, which have enhanced thermodynamic properties of selection, solvation, penetration and expansion. The ultra-low interfacial tension of SuperFluids allows complete penetration into microporous structures. As such, SFS can readily penetrate and inflate viral particles. The overfilled particles are then rapidly decompressed, and the dense-phase fluid rapidly changes into a gaseous state rupturing the virus particles at their weakest points—very much like the embolic disruption of the eardrum of a scuba diver who surfaces too rapidly. The disruption of viral structure and release of nucleic acids blocks replication and infectivity of the CFI treated viral particle. CFI has been repeatedly shown to physically disrupt viral and microbial particles (shown by TEM stains of bacteriophage virus Φ-6 before and after CFI treatment in FIG. 2). CFI is purely physical and does not use any chemicals such as formaldehyde, formalin or β-propiolactone.

While previous attempts at developing inactivated vaccines have led largely to disappointment, CFI shows great promise as a technique for developing inactivated vaccines that are both safe and protective. Previous inactivated vaccines were unsuccessful due to the degradation of the surface proteins. Techniques used to inactivate HIV and influenza have included formalin treatment, detergent disruption, exposure to psoralen and ultraviolet light and treatment with β-propiolactone. Such methods are known to denature protein, chemically modify protein and nucleic acid, disrupt macromolecular interactions and otherwise decrease the ability of the inactivated vaccine to generate an effective IR. In addition, these methods often involve potentially hazardous materials; for example, β-propiolactone is considered carcinogenic. CFI, on the other hand, does not destroy the essential native structure of proteins and can utilize non-carcinogenic or nontoxic substances, such as carbon dioxide or nitrous oxide. Because SuperFluids CFI inactivates enveloped viruses with the potential of retaining the integrity of proteins, this technology presents great promise for the development of an effective whole inactivated vaccine against infectious SARS-CoV-2 that causes COVID-19, influenza, HIV and other infectious human and animal viruses and pathogens. Embodiments of the present invention address these problems inherent in the prior art with the application of supercritical, critical or near-critical fluids, with or without polar cosolvents.

The potency and long-term sustained efficacy of the inactivated vaccine is achieved by the nanoencapsulation of the inactivated virions and pathogens into biodegradable polymer nanospheres. In an embodiment of the present invention, SuperFluids™ are also used for the nanoencapsulation of the inactivated vaccine.

In current practice, hydrophobic microspheres are manufactured using organic solvents. While the technology is highly promising, historically there have been several technical problems associated with the use of conventional organic phase solvents in polymer microencapsulation: (1) the exposure of therapeutic agent to organic solvents can adversely affect integrity of the final product; (2) large scale processing utilizes large quantities of organic solvents; (3) production is very time consuming, costly and inefficient; (4) residual toxic organic solvents in final product may elicit adverse reactions in recipients; (5) compliance with organic solvent limits established by regulatory authorities in the United States and Europe is a manufacturing challenge; and (6) these approaches have environmental waste disposal and potential contamination challenges. To circumvent these problems, SuperFluids are used to make biodegradable polymer nanospheres without the use of organic solvents.

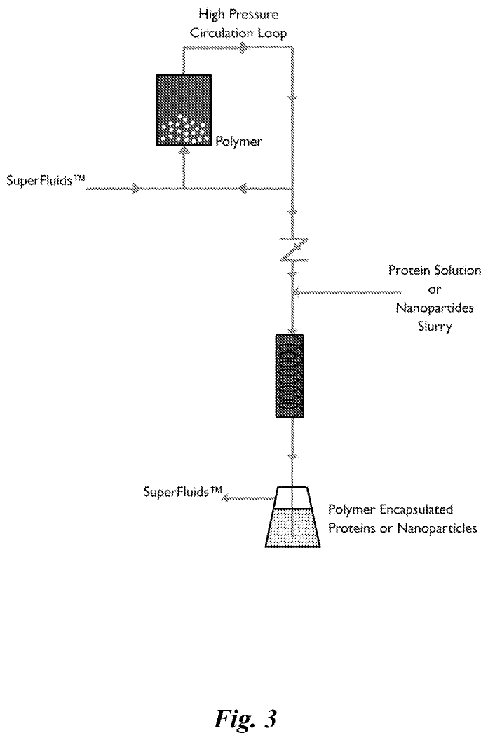

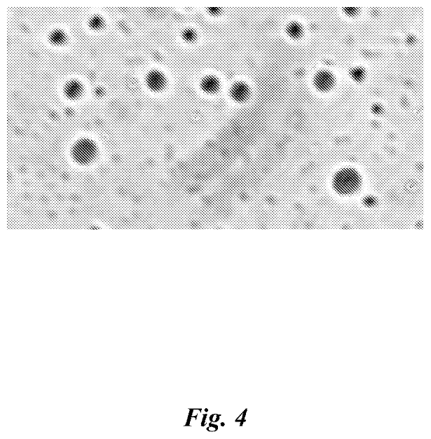

The use of SuperFluids™ or SFS, greatly reduces the processing time and costs associated with the preparation of biodegradable polymer microspheres containing vaccine products while maintaining the uniformity and integrity of the particles. Such ‘green’ technology-based fluids replace toxic organic solvents. In Aphios' SuperFluids™ polymer nanospheres [SFS-PNS]process, shown schematically in FIG. 3, a biodegradable polymer is dissolved in SuperFluids™ and decompressed through a nozzle into an aqueous solution containing the target therapeutic. The decompression creates polymer nanospheres that have simultaneously encapsulated the vaccine antigen. The safety of this approach has been validated by the use of supercritical fluids as microbicidal and virucidal technologies. We have also utilized PNS to nanoencapsulate Bryostatin-1 and make it orally bioavailable as an Alzheimer's disease therapeutic. PNS was used to target several enzymes, proteins and peptides, such as biosynthetic insulin, making it orally bioavailable in a rat model of diabetes. We have also developed polymer nanospheres encapsulating vaccine antigens such as recombinant protein antigen (rPa) for use in anthrax vaccinations (FIG. 4).

Another embodiment of the invention involves the integration of these two processes in continuous flow nanoencapsulation of inactivated influenza virions to produce a safer, stable and more effective influenza vaccine (based on longer-term antigen release). While supercritical fluids have been previously used to inactivate viruses, and supercritical fluids have been previously for encapsulation, this is the first time when these distinct levels of innovation are combined and integrated to support the development of a viral and pathogen vaccine. Sustained release vaccines achieve better humoral and cellular immune responses and immunogenicity to several immunogen, including influenza virus ones, which in addition to the immediate benefits, will increase vaccine acceptance. In addition to its benefit for influenza virus infections, this vaccination approach would be very significant in the age of the novel coronavirus pandemic and provide a new technology platform for other viral vaccines.

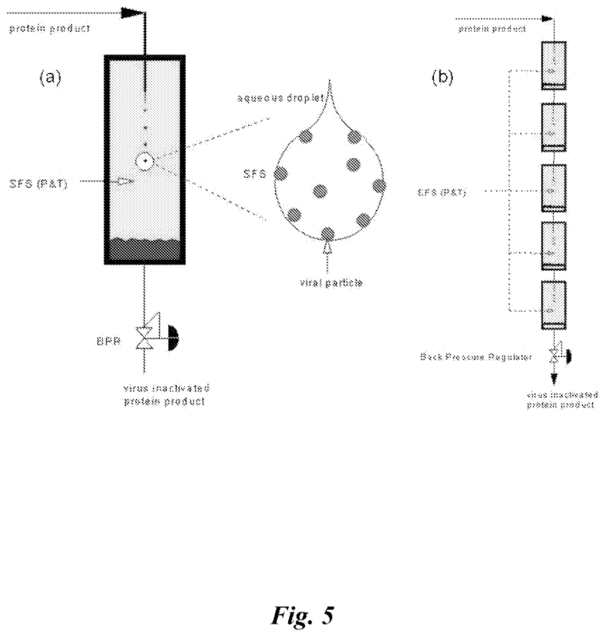

In this invention, SFS at operating pressure and temperature is first saturated with a hydrophobic, biodegradable polymer, such as pharmaceutical-grade PLGA [poly (D,L-lactide-co-glycolide) 50:50] polymer or PCL [Poly-caprolactone]. The feed stream containing virions and pathogens is injected as stream into an isobaric mixing chamber containing the polymer-enriched SFS as shown in FIG. 5a. Since the SFS is usually immiscible with the aqueous phase, virions on the surface of the droplet are more likely to be contacted and saturated with SFS than virions in the interior of the droplet. As the droplet size decreases, the probability of having more virions on the surface and the level of inactivation increases. The time required to approach the equilibrium concentration of SFS by diffusion into the interior of an aqueous droplet can be tailored by choosing the injector inner diameter, length of the mixing or drop section and flow rate. Testing in a single-stage laminar flow CFI device demonstrated that inactivation levels can be significantly increased and residence times can be significantly reduced by contacting the SuperFluids with small-diameter aqueous droplets or streams.

Volume throughput is scaled by increasing the cross-sectional area of the isobaric chamber. More significantly, inactivation levels are increased by adding stages as shown in FIG. 5b. In going from one stage to another, the surfaces of the droplets are reformed stochastically, presenting a different spectrum of droplets and virions on their surfaces to be contacted and saturated by the SFS in the isobaric chamber. Theoretically, the level of inactivation can be thus increased by the following equation:

Total Logs Inactivation≃Logs Inactivation Per Stage×Number of Stages+Logs Inactivation via BPR
. . . where BPR is the backpressure regulator that controls the final pressure reduction step (after the last stage) to atmospheric pressure

This approach confers several advantages: (1) shear forces are minimized, reducing possible damage to proteins; (2) contact of the aqueous stream with the walls of the mixer can be minimized, reducing possible protein loss; and (3) mixing geometry is simple and scalable. Volume throughput can be scaled by increasing the cross-sectional area of the isobaric mixing chamber as in chromatographic column scale-up; inactivation can be increased by adding stages as is done for improving separation efficiency in a distillation column (FIG. 5b).

We have shown that the CFI process can inactivate greater than 6 logs of several enveloped viruses in times less than 20 seconds in a single-stage laminar flow unit. We have also shown that the level of inactivation can be increased with additional process stages. Therefore, a two-stage CFI unit, which will enable more complete inactivation of enveloped viruses such as HIV, influenza and SARS-CoV-2.

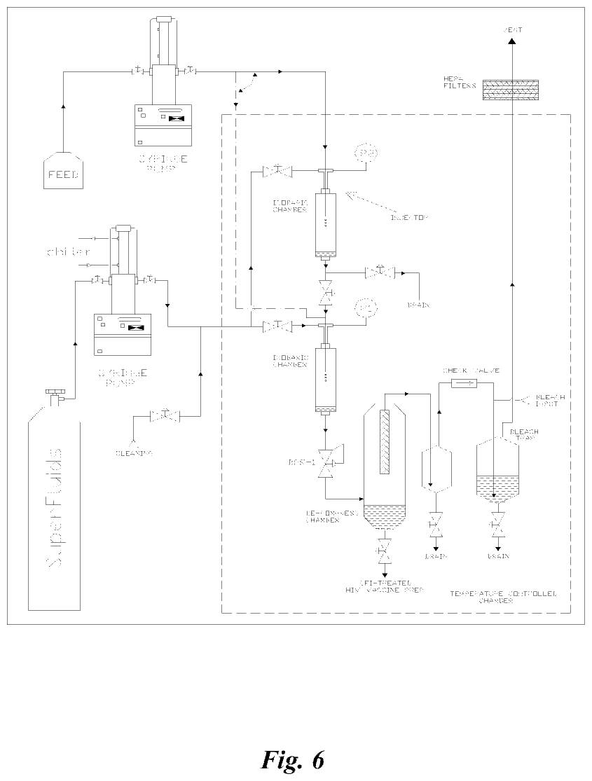

A process flow diagram (PFD) of a two-stage CFI/PNS unit (with two sequential isobaric chambers and one decompression chamber) is shown in FIG. 6 for the simultaneous inactivation and nanoencapsulation of the SARS-CoV-2 virus using polymer-enriched SuperFluids. In this process, a biodegradable polymer such as PLGA is placed in the solids chamber on the line exiting the syringe pump that is connected to a high-pressure circulation loop which is recirculated for 30 minutes to saturate the SFS with polymer. The polymer-enriched SFS is transported to the isobaric chamber wherein it contacts a feed stream containing influenza, HIV, SARS-CoV-2 or other virions and pathogens that are continuously decompressed through a nozzle into an aqueous buffer (e.g., 10% sucrose at pH 7 containing 0.1% VA (polyvinyl alcohol). As a result of decompression, the hydrophobic molecules come out of the SFS solution, thermodynamically self-assemble in an aqueous environment and polymer nanospheres are formed encapsulating inactivated virions and pathogens.

In another embodiment of this invention, the hydrophobic polymers are pre-dissolved in the SuperFluids tank or source in FIG. 6.

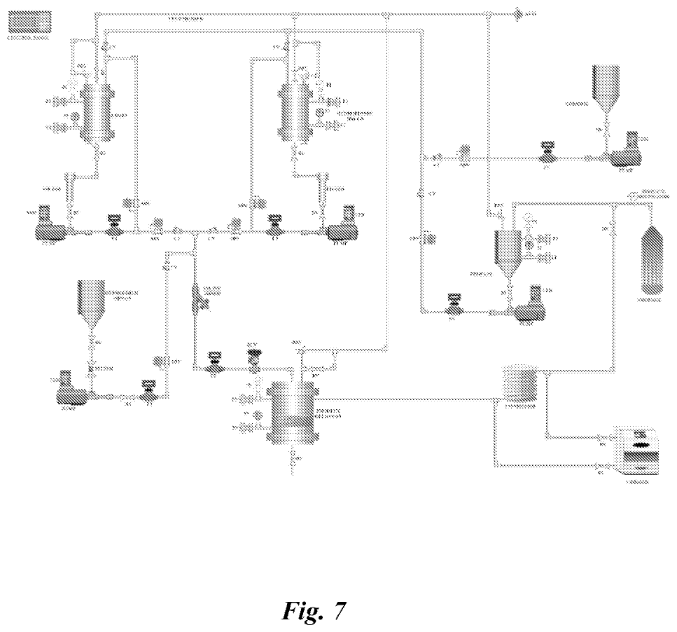

In another embodiment of the invention, shown in FIG. 7, the CFI/PNS process is scaled-up by a factor of 100 with a 10:1 turndown ratio to manufacture the vaccine nanoparticles can be manufactured following cGMP guidelines for clinical studies and commercial use. Critical scale-up process parameters and unit operations are mathematically modeled and investigated in the laboratory. Designs are then established to resolve the critical parameters using fundamental engineering principles and experimental data. We then perform detailed engineering design with piping & instrumentation (P&ID) drawings as well as detailed equipment specifications. The pilot-scale CFN unit is constructed, tested and operated. We then establish standard operating procedures (SOPs) and batch records to record manufacturing runs following cGMP guidelines.

Smaller and more tightly packed nanospheres exhibit significantly longer residence times in biological environments and show enhanced immunogenicity. Several operational conditions including temperature, nozzle size and rate of decompression strongly influence nanospheres size and distributions. Other important parameters that affect size and distribution include polymer type, nanospheres composition and virion:polymer ratios that also effect stability in a biological environment. Nanospheres of different sizes ranging from 100 to 250 (±50) nm are produced by various polymer materials, sterilized by 0.22 μm filtration and lyophilized.

Hydrophobic Biodegradable Polymers include pharmaceutical-grade PLGA [poly (D,L-actide-co-glycolide) 50:50] polymer (Resomer® RG-502, Boehringer Ingelheim KG) and PCL [Poly-caprolactone] purchased from Sigma Aldrich.

In summary, the simultaneous inactivation and nanoencapsulation of virions and pathogens are achieved by utilizing SuperFluids™ enriched with biodegradable polymers to penetrate the virions and pathogens under moderate conditions of temperature and pressure to inactivate the virions and pathogens as a result phase change from a fluid to a gas during decompression, and to nanoencapsulate the inactivated virions and pathogens in polymer nanospheres as a result of thermodynamically-driven self-assembly in an aqueous biocompatible buffer to create safe, highly-immunogenic, sustained-release nanovaccines for COVID-19, influenza, HIV and other infectious human and animal viruses and pathogens.

While this invention has been particularly shown and described with references to specific embodiments, it will be understood by those skilled in the art that various changes in form and details may be made therein without departing from the spirit and scope of the invention as defined by the appended claims.

### EXAMPLES

### Example 1: Inactivation of HIV-1 By Different SuperFluids at 3,000 psig and 22° C.

To determine the effect of different SuperFluids on HIV inactivation, supernatant from HIVΔtat-rev—infected CEM-TART cells was thawed the day of the experiment and diluted 1:10 in RPMI. Diluted virus was used immediately or kept at 4° C. A sample of diluted virus was held at the same temperature for the same time (t&T control) as that applied to the CFI unit. After the run, the tissue culture infectious dose 50 (TCID₅₀) assay for the t&T control and CFI-treated samples was begun to measure infectious virus as described above. It was noted that cells at the top dilution of virus (1:10) did not grow, and therefore were not included when calculating the TCID₅₀. Thus, the limit of detection for this assay is 2.7 logs. The Log Kill was calculated by dividing the Log TCID₅₀/ml of the t&T control by the Log TCID₅₀/ml of the CFI-Treated sample.

Table 2 show the results of eight experiments using different SuperFluids: N₂O, N₂O/CO₂ (N₂O with trace quantities of CO₂, 23 ppm), Freon-22, Propane, N₂O+CO₂ (a mixture of 95% N₂O and 5% CO₂ by volume), N₂, CO₂ and Freon-23. These results show that greater than 3.4 logs of inactivation can be achieved with SuperFluids N₂O/CO₂, while SuperFluids Propane was not able to substantially inactivate HIVΔtat-rev.

TABLE 2
Inactivation of HIV-1 By Different SuperFluids at 3,000 psig
and 22° C. In a Single-Stage Laminar Flow SuperFluids CFI Unit

| | | | | Log₁₀ | Log₁₀ | |
|---|---|---|---|---|---|---|
| | | Co- | | TCID₅₀/ml | TCID₅₀/ml | −Log₁₀ |
| Run No. | SuperFluids | Solvent | Virus | (t & T) | (CFI-treated) | Kill |
| VAC-5 | N₂O | None | HIV-1Δtat-rev | 2.8 | 2.7 | >0.1 |
| VAC-6 | N₂O/CO₂ | None | HIV-1Δtat-rev | 5.7 | 2.7 | >3.0 |
| VAC-8 | Fr-22 | None | HIV-1Δtat-rev | 5.1 | 2.7 | >2.4 |
| VAC-9 | C₃H₈ | None | HIV-1Δtat-rev | 5.0 | 4.1 | 0.9 |
| VAC-10 | N₂O | 5% CO₂ | HIV-1Δtat-rev | 5.1 | 2.7 | >2.4 |
| VAC-11 | N₂ | None | HIV-1Δtat-rev | 5.1 | 2.7 | >2.4 |
| VAC-12 | CO₂ | None | HIV-1Δtat-rev | 3.7 | 2.7 | >1.0 |
| VAC-13 | Fr-23 | None | HIV-1Δtat-rev | 3.7 | 2.7 | >1.0 |

### Example 2: Effect of Different SuperFluids CFI on HIV-1 p24

To determine the presence of a major capsid protein of HIV after treatment with SuperFluids, the amount of p24 in the t&T control and the CFI-treated samples for each SuperFluids was determined by ELISA (Table 3). Higher amounts of p24 were generally detected in the CFI-treated samples as compared to the t&T control samples.

TABLE 3
Effect of Different SuperFluids at 3,000 psig and 22° C.
on HIV-1 p24 In a Single-Stage Laminar Flow SuperFluids CFI Unit

| | | | | p24 | p24 | |
|---|---|---|---|---|---|---|
| | Super- | Co- | | [t & T] | [CFI-treated] | Δp24 |
| Run No. | Fluids | Solvent | Virus | (ng/ml) | (ng/ml) | [% Change] |
| VAC-5 | N₂O | None | HIV-1Δtat-rev | 56 | 70 | +25 |
| VAC-6 | N₂O/CO₂ | None | HIV-1Δtat-rev | 109 | 99 | −9 |
| VAC-8 | Fr-22 | None | HIV-1Δtat-rev | 120 | 112 | −7 |
| VAC-9 | C₃H₈ | None | HIV-1Δtat-rev | 146 | 175 | +20 |
| VAC-10 | N₂O | 5% CO₂ | HIV-1Δtat-rev | 107 | 82 | −23 |
| VAC-11 | N₂ | None | HIV-1Δtat-rev | 107 | 143 | +34 |
| VAC-12 | CO₂ | None | HIV-1Δtat-rev | 14 | 15 | +7 |
| VAC-13 | Fr-23 | None | HIV-1Δtat-rev | 14 | 20 | +43 |

### Example 3: CFI-Treated HIV Generates a Humoral Immune Response in Mice

To determine if CFI-treated HIV could generate a humoral immune response in mice, groups of 5 mice each were injected intraperitoneally (i.p) with either CFI-treated HIV (Experiment #2) or heat-treated HIV (68° C. for 1 hour) in incomplete Freund's adjuvant. Each mouse was injected with 1.8 μg of p24. On days 0, 14, 21 and 28 post inoculation retro orbital bleeds were performed and the sera collected. Antibody titers were measured against a purified HIV lysate in a standard ELISA. Briefly, 500 ng/well of the purified HIV lysate was coated onto wells of a 96-well plate overnight in carbonate buffer (pH=9.6). After blocking the plate with 2% BSA in PBS containing 0.02% Tween 20 (PBST) for 1 hour at 37° C., sera were diluted in PBST and 50 μl/well was added. The plate was incubated at 37° C. for 1 hour. Six half-log dilutions of each serum were tested in duplicate. After washing away unbound sera, the plate was incubated with 50 μl/well of 1:1000 alkaline phosphatase-conjugated goat anti-mouse IgG, IgA, IgM antibody for one hour at 37° C. Substrate was added and the plate incubated at room temperature in the dark for 30 minutes, after which it was read in a microplate reader at 405 nm. Positive (anti-gp41, Chessie 8) and negative controls (normal mouse serum; NMS) were included with each plate. The reciprocal of the highest dilution that gave a positive result, as determined by the mean plus two standard deviations of the negative control, was taken as the antibody titer. In cases where only one replicate was positive, the dilution was considered positive only if the average of the replicates fell above the cutoff.

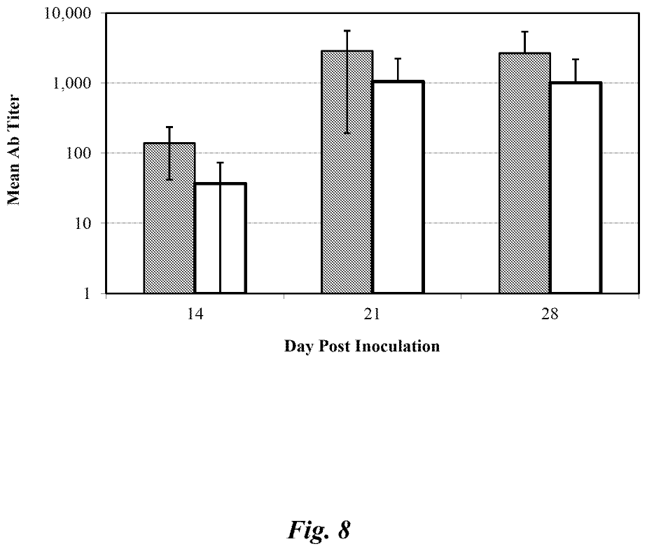

Results are presented in Table 4 and FIG. 8. The titer of antibodies was similar when mice were inoculated with either heat-treated or CFI-treated HIV (t Test p=0.49). Therefore, CFI-treated HIV is as efficient as heat-treated HIV in generating a humoral immune response.

TABLE 4
Antibody Titer in Sera from Mice Injected I.P.
with CFI-Treated and Heat-Treated HIV Particles

| | Day 0 | | Day 14 | | Day 21 | | Day 28 | |
|---|---|---|---|---|---|---|---|---|
| Mouse | Heat- | CFI- | Heat- | CFI- | Heat- | CFI- | Heat- | CFI- |
| Number | Treated | Treated | Treated | Treated | Treated | Treated | Treated | Treated |
| 1 | — | — | 330 | 33 | 3300 | 330 | 3300 | 3300 |
| 2 | — | — | 100 | 33 | 3300 | 100 | 3300 | 1000 |
| 3 | — | — | 330 | 100 | 100 | 330 | 330 | 3300 |
| 4 | — | — | 100 | — | 1000 | 1000 | 1000 | 100 |
| 5 | — | — | 33 | 33 | 1000 | 1000 | 3300 | 330 |

### Example 4: Anthrax Recombinant Protein Antigen (rPA) Nanoencapsulation

Experiments to produce a nanoencapsulated anthrax recombinant Protein Antigen (rPA) vaccine by Aphios' SuperFluids™ method used the bench-scale PNS apparatus shown in FIG. 3. Three preparations were made by varying the components and were characterized for rPA content and mean particle size. The results of the characterization are listed in Table 5.

TABLE 5
Characterization of SFS-PNP rPA Preparations

| | rPA** | Mean Particle |
|---|---|---|
| SFS-PNS # | (mg) | Size (μm) |
| rPA-01* | 5.0 | 1.54 |
| rPA-02** | 3.6 | 0.61 |
| rPA-03*** | 9.6 | 0.37 |

*rPA-01 was formed from a feed of 1.5 mg/mL rPA in PBS and SFS-PLGA in 1% PVA
**rPA-02 was formed from a feed of rPA-01 (0.25 mg/mL) and SFS-PLGA in 1% PVA
***rPA-03 was formed from a feed of rPA-01 (0.25 mg/mL) and SFS-PLGA in DI water

The data in Table 5 indicates that the particle sizes of the polymer nanospheres forned encapsulating rPA ranged from 370 nm to 1.54 μm. The data also shows that all the three preparations contained rPA indicating that the encapsulation process did not destroy the antigen. A photomicrograph of rPA-02 nanoparticles is shown as FIG. 4. The particles are mostly uniforn in size with a few variants.

### Example 5: Nanoencapsulated Anthrax Recombinant Protein Antigen (rPA) Immunization and Efficacy Testing

rPA polymeric nanospheres (rPA-01) were lyophilized and stored at 5° C. during transportation and storage. Lyophilized rPA-01 was formulated with 20% alhydrogel (v/v) in PBS to produce a final rPA concentration of 200 EEg/mL. Similarly, rPA alone was formulated with 20% alhydrogel (v/v) in PBS to produce a final rPA concentration of 200 μg/mL, as a control. Female adult A/J mice (Harlan, UK) were inununized once with a 20-μg dose in 0.1 mL intra-muscularly for either preparation (groups of 5) (Table 6). Response to immunization was monitored by bleeding the mice on day 14 and measuring IgG titers to rPA in serum samples by standard ELISA. Mice were then challenged on day 21 with 10³ MLD equivalent to 10⁶ colony forming units (cfu) of Bacillus anthracis STI strain spores intraperitoneally (IP). Survival was monitored over the subsequent 14 days.

In vivo studies were performed in England by the Defense Science and Technology Laboratory (DSTL), Porton Down, Salisbury, Wiltshire, England, part of the United Kingdom's Ministry of Defense.

TABLE 6
Serum anti-rPA Titers and Survival following Challenge

| | | IgG | | |
|---|---|---|---|---|
| Group | Formulation | (GMT*) | SEM** | Survival*** |
| 1 | rPA/alhydrogel | 0.5 | 0.12 | 5 |
| 2 | rPA-01 | 0.61 | 0.11 | 5 |
| 3 | Negative control | — | — | 0 |
| | (PBS alone) | | | |

*GMT—geometric mean titers
**SEM—standard error of the mean
***Survival (out of 5) at day 14 after challenge with 10⁶ cfu B. anthracis

The results of the preliminary study indicate the following:
1. All formulations tested were immunogenic—both encapsulated and nonencapsulated.
2. rPA-01 produced 22% higher IgG titer suggesting that it is more immunogenic than the positive control (free rPA).

All the vaccinated mice survived the challenge indicating preservation of immunogenicity for the encapsulated antigen. Results are encouraging in that rPA-01 was fully protective since this was first time that rPA had been encapsulated in biodegradable polymer nanospheres by the SFS-PNS process.

What is claimed is:

1. A method of manufacturing an encapsulated, inactivated pathogen vaccine product comprising the steps of:
(a) forming an admixture of a pathogen sample, wherein said sample contains one or more virions, and a critical, near-critical or supercritical fluid wherein said fluid can contain a polar cosolvent, and wherein said fluid contains a biodegradable, hydrophobic polymer;
(b) removing said critical, near-critical or supercritical fluid to render pathogens inactive;
(c) retaining the integrity of one or more surface protein of said pathogen; and
(d) nanoencapsulating said inactivated pathogen to form a processed encapsulated inactivated pathogen vaccine product.

2. The method of claim 1, wherein said processed pathogen vaccine product exhibits a 2.7 log reduction in pathogen activity compared to said virus sample.

3. The method of claim 1, wherein said critical, near-critical or supercritical fluid is at a temperature in the range of 0° C. to 100° C.

4. The method of claim 3, wherein said critical, near-critical or supercritical fluid has a temperature that does not exceed 60° C.

5. The method of claim 4, wherein said critical, near-critical or supercritical fluid has a temperature range of range of 4° C. to 40° C.

6. The method of claim 1 wherein said admixture is formed and maintained at a pressure of 0.75 to 20.0 times the critical pressure of one or more gases comprising the critical, near-critical or supercritical fluid.

7. The method of claim 1, wherein said critical, near-critical or supercritical fluid is selected from one or more of the gases of the group consisting of fluorocarbons, alkanes, binary gases, and a combination thereof.

8. The method of claim 7, wherein said critical, near-critical or supercritical fluid is selected from one or more of the gases of the group consisting of nitrous oxide, chlorodifluoromethane, propane and carbon dioxide, and a combination thereof.

9. The method of claim 1, wherein said critical, near-critical or supercritical fluid further comprises one or more said polar modifiers selected from the group consisting of ethanol, methanol, acetone and ethylene glycol, and a combination thereof.

10. The method of claim 8, wherein said critical, near-critical or supercritical fluid is chlorodifluoromethane at approximately 10° C. to 60° C. and 800 to 5,000 psig.

11. The method of claim 8, wherein critical, near-critical or supercritical fluid is nitrous oxide at approximately 12° C. to 30° C. and 1,000 to 3,000 psig.

12. The method of claim 8, wherein critical, near-critical or supercritical fluid is a mixture of nitrous oxide and carbon dioxide at approximately 12° C. to 30° C. and 1,000 to 3,000 psig.

13. The method of claim 12, wherein said mixture is primarily nitrous oxide with approximately 10 to 1,000 parts per million carbon dioxide.

14. The method of claim 1, wherein said pathogen is an enveloped virus.

15. The method of claim 14, wherein said enveloped virus is SARS-CoV-2, influenza and HIV-1.

16. The method of claim 1, wherein said pathogen is a nonenveloped virus.

17. The method of claim 1, wherein said pathogen is a bacteria.

18. An apparatus for making an encapsulated, inactivated pathogen vaccine product, comprising: (i) an isobaric mixing chamber; (ii) a circulation loop in fluid communication with an isobaric mixing chamber for forming a solution of a hydrophobic, biodegradable polymer in a supercritical, critical or near critical fluid which can contain a polar cosolvent; (iii) a pathogen feed stream in fluid communication with the isobaric mixing chamber for introducing said pathogen feed stream into the isobaric mixing chamber; (iv) an injection nozzle in fluid communication with the isobaric mixing chamber for receiving the mixture and releasing the mixture as a stream into a decompression buffer; and (v) a decompression vessel in fluid communication with the injection nozzle for holding a decompression buffer and receiving the mixture as a stream, wherein the pathogen particles are inactivated and wherein polymer nanospheres are formed in the decompression buffer, encapsulating the inactivated pathogen particles.

19. The apparatus of claim 18, further including a second isobaric chamber arranged in tandem with the said isobaric chamber to form two tandem stages, so that processing of the pathogen particles proceeds to the second chamber to increase the efficiency of the pathogen particle inactivation.

20. The apparatus of claim 18, further including a plurality of isobaric chambers arranged in tandem with the said isobaric chamber, so that processing of pathogen particles proceeds from one isobaric chamber to the next to increase the efficiency of the pathogen particle inactivation.

21. An apparatus for making an encapsulated, inactivated pathogen vaccine product, comprising: (i) an isobaric mixing chamber; (ii) a solution of a hydrophobic, biodegradable polymer in a supercritical, critical or near critical fluid which can contain a polar cosolvent; (iii) a pathogen feed stream in fluid communication with the isobaric mixing chamber for introducing said pathogen feed stream into the isobaric mixing chamber; (iv) an injection nozzle in fluid communication with the isobaric mixing chamber for receiving the mixture and releasing the mixture as a stream into a decompression buffer; and (v) a decompression vessel in fluid communication with the injection nozzle for holding a decompression buffer and receiving the mixture as a stream, wherein the pathogen particles are inactivated and wherein polymer nanospheres are formed in the decompression buffer, encapsulating the inactivated pathogen particles.

22. The apparatus of claim 21, further including a second isobaric chamber arranged in tandem with the said isobaric chamber to form two tandem stages, so that processing of the pathogen particles proceeds to the second chamber to increase the efficiency of the pathogen particle inactivation.

23. The apparatus of claim 21, further including a plurality of isobaric chambers arranged in tandem with the said isobaric chamber, so that processing of pathogen particles proceeds from one isobaric chamber to the next to increase the efficiency of the pathogen particle inactivation.

24. A sustained release vaccine product comprised of an inactivated pathogen, wherein the pathogen is inactivated by a critical, near-critical or supercritical fluid wherein said fluid can contain a polar cosolvent, and wherein said fluid contains a biodegradable, hydrophobic polymer.

25. The sustained release vaccine product of claim 24, wherein said polymer is selected from a group of biodegradable, hydrophobic polymers comprising of poly (D,L-lactide-co-glycolide), polycaprolactone, and a combination thereof.

26. The sustained release vaccine product of claim 24, wherein the preferred said polymer is poly (D,L-lactide-co-glycolide).

27. The sustained release vaccine product of claim 24, wherein said pathogen is an enveloped virus.

28. The sustained release vaccine product of claim 27, wherein said virus is HIV-1.

29. The sustained release vaccine product of claim 27, wherein said virus is influenza.

30. The sustained release vaccine product of claim 27, wherein said virus is SARS-CoV-2.

31. The sustained release vaccine product of claim 24, wherein said pathogen is a nonenveloped virus.

32. The sustained release vaccine product of claim 24, wherein said pathogen is a bacteria.
